(19) United States
(12) Patent Application Publication
(10) Pub. No.: US 2026/0306632 A1
(43) Pub. Date: Oct. 8, 2026

(54) NANOPARTICLES FOR PHOTOIMMUNE THERAPY COMPRISING PHOTOSENSITIZER AND ADJUVANT, AND USAGE THEREOF

(71) Applicants: KOREA INSTITUTE OF SCIENCE AND TECHNOLOGY, Seoul (KR); SAMSUNG LIFE PUBLIC WELFARE FOUNDATION, Seoul (KR)

(72) Inventors: Se Hoon KIM, Seoul (KR); Jin Kyoung YANG, Seoul (KR); Assel SEITKAZINA, Seoul (KR); Ana Paula Mesquita SOUZA, Seoul (KR); Yu Ri CHO, Seoul (KR); Han-sin JEONG, Seoul (KR)

(73) Assignees: KOREA INSTITUTE OF SCIENCE AND TECHNOLOGY, Seoul (KR); SAMSUNG LIFE PUBLIC WELFARE FOUNDATION, Seoul (KR)

(21) Appl. No.: 19/143,705

(22) PCT Filed: Nov. 9, 2023
(86) PCT No.: PCT/KR2023/018001

§ 371 Date: Jun. 23, 2026

(30) Foreign Application Priority Data
Dec. 26, 2022 (KR) 10-2022-0184928

(51) Int. Cl. A61K 41/00; A61K 31/4745; A61K 39/00; A61K 39/395; A61K 47/54; A61K 47/69; A61P 35/00

(52) U.S. Cl. CPC A61K 41/0057; A61K 31/4745; A61K 39/3955; A61K 47/542; A61K 47/545; A61K 47/6907; A61K 47/6935; A61P 35/00; A61K 2039/505

(57) ABSTRACT

The present invention relates to nanoparticles, a pharmaceutical composition comprising same for preventing or treating cancer or a cell hyperplasia disorder, and the like, the nanoparticles comprising: a methylene blue-organic acid complex; a cationic adjuvant-organic acid complex; and an amphiphilic polymer, wherein the nanoparticles are an agent having excellent drug properties and permeability, and are composed only of substances in clinical use so as to have low barriers to clinical entry and high potential for commercialization. In addition, the nanoparticles make it possible to have photodynamic therapy and immunotherapy, simultaneously, and thus may be utilized in the field of preventing, ameliorating, or treating cancer or a cell hyperplasia disorder.

### TECHNICAL FIELD

The present invention relates to nanoparticles for photoimmunotherapy including a photosensitizer and an adjuvant, and usage thereof.

### BACKGROUND ART

Conventional cancer treatment methods have mainly consisted of surgical operation, chemotherapy, radiotherapy, and the like, and these methods not only have extremely limited therapeutic effects but also damage normal tissues, causing serious side effects, thus requiring the development of new treatment methods.

Photodynamic therapy is based on the principle that photosensitizers exposed to light generate singlet oxygen and other reactive oxygen species. This technology is attracting attention because it enables disease-selective treatment with minimally invasive methods compared to existing technologies such as surgery, chemotherapy, and radiation therapy. In addition, photodynamic therapy may induce anticancer immune responses after treatment, so therapeutic effects against residual cancer can also be expected. However, such immune effects are at a weak level to prevent cancer metastasis or recurrence. Therefore, there is a need to develop combination therapies that can reinforce such effects.

Immunotherapy is a technology that treats cancer by activating the human immune system, and when anticancer immune action is induced, distinct effects can be expected during treatment, tumor suppression effects are shown even for cancer metastasis, and long-term tumor suppression (complete cure) can be achieved, and therefore active clinical application is being conducted recently. However, immunotherapeutic agents cause effective anticancer immune responses only in some (about ¼ to ⅓) cancer patients, and may cause side effects due to excessive immune responses depending on selectivity and sensitivity, so technologies that promote anticancer immune responses or selectively induce immune responses are needed. Therefore, for anticancer treatment, efficient delivery selectively to cancer sites must be possible, so nanoparticulation technology of adjuvants is required.

Under this background, the present inventors confirmed that cancer treatment efficiency can be increased by simultaneously performing photodynamic therapy and immunotherapy using nanoparticles including methylene blue and imiquimod that are in clinical use, and completed the present invention.

### DISCLOSURE OF THE INVENTION

TECHNICAL GOALS

An object of the present invention is to provide a nanoparticle including a methylene blue-organic acid complex; a cationic adjuvant-organic acid complex; and an amphiphilic polymer.

Another object of the present invention is to provide a pharmaceutical composition for preventing or treating cancer or a cell hyperplasia disorder, including the nanoparticle as an effective ingredient.

Another object of the present invention is to provide a topical dosage form for preventing or treating cancer or a cell hyperplasia disorder, including the nanoparticle as an effective ingredient.

Another object of the present invention is to provide a method of preparing the nanoparticle.

However, technical goals to be achieved are not limited to those described above, and other goals not mentioned above are clearly understood by one of ordinary skill in the art from the following description.

### Technical Solutions

To solve the above problems, the present invention provides a nanoparticle including a methylene blue-organic acid complex; a cationic adjuvant-organic acid complex; and an amphiphilic polymer.

As one embodiment of the present invention, the cationic adjuvant may be an imidazoquinoline compound including an amine functional group.

As one embodiment of the present invention, the imidazoquinoline compound including the amine functional group may be any one or more selected from imiquimod, resiquimod, gardiquimod, and combinations thereof, but is not limited thereto, and may preferably be imiquimod.

As another embodiment of the present invention, the organic acid may be any one or more selected from the group consisting of propionic acid, butyric acid, pentanoic acid, hexanoic acid, heptanoic acid, octanoic acid, oleic acid, linoleic acid, linolenic acid, stearic acid, palmitic acid, salicylic acid, cholic acid, deoxycholic acid, lithocholic acid, and combinations thereof, but is not limited thereto, and may preferably be a fatty acid, more preferably oleic acid or a salt thereof.

As another embodiment of the present invention, the amphiphilic polymer may be any one or more selected from the group consisting of polyoxyethylene-polyoxypropylene (POE-POP) block copolymer, polyethylene oxide-polypropylene oxide-polyethylene oxide (PEO-PPO-PEO) block copolymer, polylactic acid (PLA), polyglycolic acid (PGA), block copolymer of poly(lactic-co-glycolic acid) (PLGA) and polyethylene glycol (PEG), and combinations thereof, but is not limited thereto, and may preferably be polyethylene oxide-polypropylene oxide-polyethylene oxide (PEO-PPO-PEO) block copolymer, polyoxyethylene-polyoxypropylene block copolymer, more preferably Pluronic polymer, and may be Pluronic F-127.

As another embodiment of the present invention, the methylene blue-organic acid complex and the cationic adjuvant-organic acid complex may be encapsulated inside amphiphilic polymer micelles.

As another embodiment of the present invention, the methylene blue-organic acid complex may be methylene blue physically modified with a fatty acid.

As another embodiment of the present invention, the cationic adjuvant-organic acid complex may be a cationic adjuvant physically modified with a fatty acid.

In addition, the present invention provides a pharmaceutical composition for preventing or treating cancer or a cell hyperplasia disorder, including the nanoparticle as an effective ingredient.

As one embodiment of the present invention, the nanoparticle may be used simultaneously for photodynamic therapy and immunotherapy.

As another embodiment of the present invention, the nanoparticle may activate dendritic cells to selectively kill cancer cells.

As another embodiment of the present invention, the cancer may be any one or more selected from the group consisting of skin cancer, breast cancer, uterine cancer, esophageal cancer, gastric cancer, brain tumor, colon cancer, rectal cancer, colorectal cancer, lung cancer, ovarian cancer, cervical cancer, endometrial cancer, vulvar cancer, kidney cancer, blood cancer, pancreatic cancer, pancreatic endocrine tumor, prostate cancer, testicular cancer, nasopharyngeal cancer, laryngeal cancer, head and neck cancer, thyroid cancer, liver cancer, bladder cancer, thymoma, mesothelioma, osteosarcoma, lymphoma, leukemia, thymic cancer, urethral cancer, bronchial cancer, gallbladder cancer, penile cancer, ureteral cancer, renal cell carcinoma, non-Hodgkin's lymphoma, myelodysplastic syndrome, multiple myeloma, plasma cell tumor, pediatric cancer, and combinations thereof, but is not limited thereto.

As another embodiment of the present invention, the cell hyperplasia disorder may be any one or more selected from the group consisting of acral lentiginous melanoma, actinic keratosis, adenocarcinoma, adenoid cystic carcinoma, adenoma, adenosarcoma, adenosquamous carcinoma, astrocytic tumor, glucagonoma, hemangioblastoma, hemangioendothelioma, hemangioma, hepatic adenoma, hepatic adenomatosis, hepatocellular carcinoma, insulinoma, intraepithelial neoplasia, intraepithelial squamous cell neoplasia, invasive squamous cell carcinoma, large cell carcinoma, leiomyosarcoma, lentigo maligna melanoma, malignant melanoma, malignant mesothelioma, medulloblastoma, medulloepithelioma, and combinations thereof, but is not limited thereto.

As another embodiment of the present invention, the pharmaceutical composition may further include an immune anticancer agent. Preferably, the pharmaceutical composition may further include an immune checkpoint inhibitor (ICI).

As yet another embodiment of the present invention, the immune anticancer agent may be any one or more selected from the group consisting of anti-PD-L1 (programmed death-ligand 1) antibody, anti-CTLA-4 (cytotoxic T-lymphocyte antigen-4) antibody, anti-PD-1 (programmed death-1) antibody, and combinations thereof, but is not limited thereto.

Specifically, the anti-PD-L1 antibody may be avelumab, atezolizumab, durvalumab, and the like, the anti-CTLA-4 antibody may be tremelimumab, ipilimumab, and the like, and the anti-PD-1 antibody may be nivolumab, pembrolizumab, and the like, but is not limited thereto.

In addition, the present invention provides a method of preventing or treating cancer or a cell hyperplasia disorder, including administering the nanoparticle to a subject.

In addition, the present invention provides use of the nanoparticle for the manufacture of a medicament for preventing or treating cancer or a cell hyperplasia disorder.

In addition, the present invention provides a cosmetic composition for preventing or improving cancer or a cell hyperplasia disorder, including the nanoparticle as an effective ingredient.

In addition, the present invention provides a food composition for preventing or improving cancer or a cell hyperplasia disorder, including the nanoparticle as an effective ingredient.

In addition, the present invention provides a topical dosage form for preventing or treating cancer or a cell hyperplasia disorder, including the nanoparticle as an effective ingredient.

In addition, the present invention provides a method of preparing nanoparticles, including the following steps:
(1) adding an organic acid to methylene blue to form a methylene blue-organic acid complex;
(2) adding an organic acid to a cationic adjuvant to form a cationic adjuvant-organic acid complex;
(3) adding the methylene blue-organic acid complex formed in step (1) to an amphiphilic polymer and performing ultrasonic treatment to form a thin film;
(4) rehydrating the thin film formed in step (3) with the cationic adjuvant-organic acid complex formed in step (2) and performing ultrasonic treatment; and
(5) encapsulating the mixture of step (4) to prepare nanoparticles.

As one embodiment of the present invention, the cationic adjuvant may be an imidazoquinoline compound including an amine functional group, preferably may be any one or more selected from imiquimod, resiquimod, gardiquimod, and combinations thereof, and most preferably may be imiquimod.

As another embodiment of the present invention, the organic acid may be any one or more selected from the group consisting of propionic acid, butyric acid, pentanoic acid, hexanoic acid, heptanoic acid, octanoic acid, oleic acid, linoleic acid, linolenic acid, stearic acid, palmitic acid, salicylic acid, cholic acid, deoxycholic acid, lithocholic acid, and combinations thereof, but is not limited thereto, and may preferably be a fatty acid, more preferably oleic acid or a salt thereof.

As another embodiment of the present invention, the amphiphilic polymer may be any one or more selected from the group consisting of polyoxyethylene-polyoxypropylene (POE-POP) block copolymer, polyethylene oxide-polypropylene oxide-polyethylene oxide (PEO-PPO-PEO) block copolymer, polylactic acid (PLA), polyglycolic acid (PGA), block copolymer of poly(lactic-co-glycolic acid) (PLGA) and polyethylene glycol (PEG), and combinations thereof, but is not limited thereto, and may preferably be polyoxyethylene-polyoxypropylene block copolymer, more preferably Pluronic polymer, and may be Pluronic F-127.

As another embodiment of the present invention, the methylene blue-organic acid complex and the cationic adjuvant-organic acid complex may be encapsulated inside amphiphilic polymer micelles.

As another embodiment of the present invention, the methylene blue-organic acid complex may be methylene blue physically modified with a fatty acid.

As another embodiment of the present invention, the cationic adjuvant-organic acid complex may be a cationic adjuvant physically modified with a fatty acid.

As another embodiment of the present invention, step (2) may be forming a cationic adjuvant-organic acid complex by adding a salt of an organic acid to a protonated cationic adjuvant. The protonated cationic adjuvant may be prepared by dissolving a cationic adjuvant in water to which HCl has been added.

As another embodiment of the present invention, step (2) may be adding an organic acid to a cationic adjuvant in a 1:1 equivalent ratio.

### Effects of the Invention

The nanoparticles according to one embodiment of the present invention have anticancer immune effects by inducing activation of dendritic cells in vivo. In addition, the nanoparticles have anticancer therapeutic effects by inducing activation of DAMP signals through photoimmunotherapy. The nanoparticles can enhance therapeutic effects not only in primary tumors but also in distant tumors through combination therapy of photodynamic therapy and immunotherapy. That is, the nanoparticles can be effectively used for cancer metastasis and recurrence.

The nanoparticles according to one embodiment of the present invention have excellent drug properties and permeability as methylene blue and a cationic adjuvant are respectively physically modified with organic acids. In addition, according to one embodiment of the present invention, solubility and loading capacity can be increased by encapsulating a methylene blue-organic acid complex and a cationic adjuvant-organic acid complex inside amphiphilic polymer micelles.

The nanoparticles according to one embodiment of the present invention are composed only of substances in clinical use such as methylene blue and imiquimod, and thus are formulations with low barriers to clinical entry and high potential for commercialization. In addition, the nanoparticles can increase cancer treatment efficiency because photodynamic therapy and immunotherapy are simultaneously possible.

The nanoparticles according to one embodiment of the present invention can increase anticancer immunity by increasing cytokines by further including inhibitors of immune checkpoint inhibitors such as anti-PD-L1 antibodies.

The effects of the nanoparticles according to one embodiment of the present invention and the compositions for cancer prevention or treatment including the same are not limited to those mentioned above, and other effects not mentioned will be clearly understood by those skilled in the art from the description below.

### BRIEF DESCRIPTION OF DRAWINGS

FIG. 1 confirms the dispersibility of nanoparticles (ImoMB) prepared after obtaining imiquimod-oleic acid complex (ImO) according to one embodiment of the present invention. Method 1 is obtaining ImO by dissolving imiquimod in water to which HCl has been added and then adding water in which sodium oleate is dissolved, and Method 2 is obtaining ImO by adding oleic acid to water in which imiquimod is dispersed.

FIG. 2 shows a schematic diagram of the nanoparticles of the present invention (ImoMB) and nanoparticles in which imiquimod was not modified with oleic acid (ImiMB).

FIG. 3 shows results comparing the solubility of free imiquimod and imiquimod-oleic acid complex (ImO), where FIG. 3A shows the solubility of free imiquimod and FIG. 3B shows the solubility of ImO.

FIG. 4 shows the cytotoxicity of a mixture of nanoparticles not loaded with imiquimod (NanoMB) and imiquimod-oleic acid complex (ImO) nanoparticles (ImO-F127).

FIG. 5 shows characteristics of the nanoparticles of the present invention (ImoMB), where FIG. 5A is a transmission electron microscopy (TEM) image of the nanoparticles, FIG. 5B is a result of measuring the size of the nanoparticles, FIG. 5C shows the absorbance of the nanoparticles, imiquimod-oleic acid complex (ImO), and Pluronic F-127, and FIG. 5D shows the absorbance of the nanoparticles and nanoparticles in which imiquimod was not modified with oleic acid (ImiMB).

FIG. 6 confirms the phototoxicity of the nanoparticles of the present invention (ImoMB) in head and neck cancer cell lines SCC7 and FADU.

FIG. 7 shows damage-associated molecular patterns (DAMP) expression upon light irradiation of the nanoparticles of the present invention (ImoMB) and nanoparticles not loaded with imiquimod (NanoMB), where FIG. 7A shows ATP concentration, FIG. 7B shows HSP90 intensity, FIG. 7C shows calreticulin (CRT)+ cells, and FIG. 7D shows HMGB1 intensity.

FIG. 8 shows evaluation of dendritic cell activation function of the nanoparticles of the present invention (ImoMB), where FIG. 8A shows results confirming the degree of maturation of immature dendritic cells obtained from bone marrow cells isolated from mice, FIG. 8B from the lymph of male mice, and FIG. 8C from the spleen of male mice. At this time, LPS was used as a positive control, and a group treated with nanoparticles not loaded with imiquimod (NanoMB), a group treated with a simple mixture of NanoMB and free imiquimod, and a free imiquimod alone-treated group were used as comparative examples.

FIG. 9 shows photoimmune anticancer effects of the nanoparticles of the present invention (ImoMB) in an animal model. At this time, PBS was used as a control group, and a group treated with nanoparticles not loaded with imiquimod (NanoMB), a group treated with a simple mixture of NanoMB and free imiquimod, and a free imiquimod alone-treated group were used as comparative examples.

FIG. 10 shows secondary tumor growth inhibition effects of the nanoparticles of the present invention (ImoMB). At this time, a group treated with nanoparticles in which imiquimod was not modified with oleic acid (ImiMB) was used as a comparative example.

FIG. 11 confirms changes in tumor size when the nanoparticles of the present invention (ImoMB) and anti-PD-L1 antibody, which is an immune checkpoint inhibitor, were administered in combination.

FIG. 12 confirms cytokine expression in serum when the nanoparticles of the present invention (ImoMB) and anti-PD-L1 antibody, which is an immune checkpoint inhibitor, were administered in combination.

### BEST MODE FOR CARRYING OUT THE INVENTION

The present inventors have confirmed that nanoparticles including a methylene blue-organic acid complex and a cationic adjuvant-organic acid complex, in which methylene blue and a cationic adjuvant are each physically modified with an organic acid, have excellent solubility, loading capacity, and pharmacological properties, and that the nanoparticles can be used simultaneously for photodynamic therapy and immunotherapy, thereby enhancing the efficacy of cancer treatment.

In particular, it was confirmed that the nanoparticles of the present invention (i) have a much superior anticancer effect compared to nanoparticles not loaded with a cationic adjuvant, (ii) have high solubility and loading capacity compared to nanoparticles in which the cationic adjuvant is encapsulated together with a methylene blue-organic acid complex in a free cationic adjuvant state without being modified with an organic acid, and (iii) do not have toxicity to normal cells, unlike a mixture of methylene blue-organic acid complex nanoparticles and cationic adjuvant-organic acid complex nanoparticles.

Accordingly, the present invention provides nanoparticles including a methylene blue-organic acid complex; a cationic adjuvant-organic acid complex; and an amphiphilic polymer.

In the present invention, “methylene blue” may be used as a photosensitizer in photodynamic therapy. Methylene blue is a positively charged dye that is very difficult to introduce into cells by itself, but according to one embodiment of the present invention, methylene blue forms a stable methylene blue-organic acid complex with an organic acid by electrostatic force and hydrophobic interactions in molecular structure, thereby exhibiting high solubility in solvents.

In the present invention, “adjuvant” refers to any substance that enhances a host's specific or non-specific, cellular and humoral immune response, and has recently attracted much interest as immune enhancement has been utilized in the treatment of infections or tumors. Preparations used to enhance immunity are classified into general immune enhancers and specific immune enhancers according to their specificity, and can also be classified into active immune enhancers and passive immune enhancers according to their function. The mechanisms of action of adjuvants, which enhance various immune responses occurring in vivo without immunogenicity, vary depending on the adjuvant. To enumerate a few: first, adjuvants cause a depot effect on antigens; second, adjuvants activate macrophages to increase the expression of antigens on their surface, thereby improving the presentation efficiency to lymphocytes; third, adjuvants exhibit specific effects on lymphocytes; and finally, adjuvants demonstrate antitumor effects.

In the present invention, the adjuvant may be a cationic adjuvant, and the cationic adjuvant may be an imidazoquinoline compound containing an amine functional group. The imidazoquinoline may be 1H-imidazo[4,5-c]quinoline, and the amine functional group may be substituted at the 4th carbon position of the 1H-imidazo[4,5-c]quinoline.

More specifically, the imidazoquinoline compound including the amine functional group may be any one or more selected from imiquimod, resiquimod, gardiquimod, and combinations thereof, and preferably may be imiquimod.

In the present invention, “imiquimod” is an adjuvant that stimulates the patient's immune system to secrete various chemical substances such as interferon and interleukin-12. According to one embodiment of the present invention, a salt of protonated imiquimod and oleic acid forms a stable imiquimod-oleic acid complex through ionic bonding, thereby exhibiting high solubility in a solvent.

In the present invention, “organic acid” means an organic compound that generally has acidic properties, and includes fatty acids and bile acids. The organic acid may be any one or more selected from the group consisting of propionic acid, butyric acid, pentanoic acid, hexanoic acid, heptanoic acid, octanoic acid, oleic acid, linoleic acid, linolenic acid, stearic acid, palmitic acid, salicylic acid, cholic acid, deoxycholic acid, lithocholic acid, and combinations thereof, and may preferably be a fatty acid, more preferably oleic acid or a salt thereof.

In the present invention, “fatty acid” means a carboxylic acid or a salt thereof, and may be a long-chain saturated or unsaturated monocarboxylic acid, and preferably may be an oleic acid or a salt thereof.

In the present invention, the “amphiphilic polymer” is a compound having both a hydrophilic portion and a hydrophobic portion, and any polymer that can form a micelle structure by assembling molecules can be used without limitation. When polymer micelles are formed in a hydrophilic solvent, the hydrophobic portions of the molecules gather at the center to form a core, and the hydrophilic portions form an outer portion that contacts water. In an aqueous solvent, hydrophobic substances such as oil become positioned in the inner part of micelles, thereby becoming stabilized and dissolved in the solvent.

For the amphiphilic polymer, an anionic amphiphilic polymer having carboxylate, sulfonate, or phosphate structures as hydrophilic groups, a cationic amphiphilic polymer containing quaternary ammonium salts as hydrophilic groups, an amphiphilic polymer having both anionic sites and cationic sites within the molecule, an amphiphilic polymer having non-electrolytes, i.e., non-ionizable hydrophilic portions, as hydrophilic parts, and the like, may be used, and the amphiphilic polymer may be any one or more selected from the group consisting of polyoxyethylene-polyoxypropylene (POE-POP) block copolymer, polyethylene oxide-polypropylene oxide-polyethylene oxide (PEO-PPO-PEO) block copolymer, polylactic acid (PLA), polyglycolic acid (PGA), block copolymer of poly(lactic-co-glycolic acid) (PLGA) and polyethylene glycol (PEG), and combinations thereof, but is not limited thereto. Preferably, the amphiphilic polymer may be polyethylene oxide-polypropylene oxide-polyethylene oxide (PEO-PPO-PEO) block copolymer, polyoxyethylene-polyoxypropylene block copolymer, more preferably Pluronic polymer, and may be Pluronic F-127.

According to one embodiment of the present invention, the methylene blue-organic acid complex and the cationic adjuvant-organic acid complex are encapsulated inside amphiphilic polymer micelles, whereby the nanoparticles of the present invention can self-assemble.

In addition, the present invention provides a pharmaceutical composition for preventing or treating cancer or a cell hyperplasia disorder, including the nanoparticles as an effective ingredient.

In the present invention, “prevention” means any act of inhibiting or delaying the occurrence, spread or recurrence of the disease by administering the composition of the present invention, and “treatment” means any act of improving or beneficially changing the symptoms of the disease by administering the composition of the present invention.

In the present invention, “pharmaceutical composition” means a composition manufactured for the purpose of preventing or treating the above-described disease, and can be formulated and used in various forms according to conventional methods. For example, the pharmaceutical composition can be formulated into oral dosage forms such as powder, granules, tablets, capsules, suspensions, emulsions, and syrups, and can be formulated and used in the form of external preparations, suppositories, and sterile injectable solutions.

In the present invention, “including as an effective ingredient” means that the corresponding component is included in an amount necessary or sufficient to realize a desired biological effect. In actual application, the determination of the amount included as an effective ingredient is an amount for treating a target disease, and may be determined by considering factors that do not cause other toxicity, and may vary according to various factors such as, for example, the disease or condition being treated, the form of the composition being administered, the size of the subject, or the severity of the disease or condition. A person skilled in the art to which the present invention pertains can empirically determine the effective amount of an individual composition without undue experimentation.

In addition, the pharmaceutical composition of the present invention may, depending on each formulation, additionally contain one or more pharmaceutically acceptable carriers in addition to the effective ingredients described above.

The pharmaceutically acceptable carrier may be saline solution, sterile water, Ringer's solution, buffered saline, dextrose solution, maltodextrin solution, glycerol, ethanol, and mixtures of one or more of these components, and may further include other conventional additives such as antioxidants, buffers, and bacteriostatic agents, as needed. In addition, diluents, dispersants, surfactants, binders and lubricants may be additionally added to formulate the composition into injectable formulations such as aqueous solutions, suspensions and emulsions, pills, capsules, granules or tablets. Furthermore, the composition may be preferably formulated for each disease or ingredient using an appropriate method in the art or a method disclosed in Remington's Pharmaceutical Science (Mack Publishing Company, Easton PA).

The composition of the present invention may be administered orally or parenterally in a pharmaceutically effective amount according to a desired method, and the term “pharmaceutically effective amount” of the present invention means an amount that is sufficient to treat a disease with a reasonable benefit/risk ratio applicable to medical treatment and does not cause side effects, and the effective dose level may be determined according to factors including the patient's health condition, severity, activity of the drug, sensitivity to the drug, administration method, administration time, administration route and excretion rate, treatment period, drugs used in combination or concurrently, and other factors well known in the medical field.

The nanoparticles of the present invention exhibit a therapeutic effect by specifically targeting a site of a cell hyperplasia proliferation disease such as cancer or a tumor, and the cancer that can be prevented or treated by the present invention may be any one or more selected from the group consisting of skin cancer, breast cancer, uterine cancer, esophageal cancer, gastric cancer, brain tumor, colon cancer, rectal cancer, colorectal cancer, lung cancer, ovarian cancer, cervical cancer, endometrial cancer, vulvar cancer, kidney cancer, blood cancer, pancreatic cancer, pancreatic endocrine tumor, prostate cancer, testicular cancer, nasopharyngeal cancer, laryngeal cancer, head and neck cancer, thyroid cancer, liver cancer, bladder cancer, thymoma, mesothelioma, osteosarcoma, lymphoma, leukemia, thymic cancer, urethral cancer, bronchial cancer, gallbladder cancer, penile cancer, ureteral cancer, renal cell carcinoma, non-Hodgkin's lymphoma, myelodysplastic syndrome, multiple myeloma, plasma cell tumor, pediatric cancer, and combinations thereof, but is not limited thereto. The cell hyperplasia disorder may be any one or more selected from the group consisting of acral lentiginous melanoma, actinic keratosis, adenocarcinoma, adenoid cystic carcinoma, adenoma, adenosarcoma, adenosquamous carcinoma, astrocytic tumor, glucagonoma, hemangioblastoma, hemangioendothelioma, hemangioma, hepatic adenoma, hepatic adenomatosis, hepatocellular carcinoma, insulinoma, intraepithelial neoplasia, intraepithelial squamous cell neoplasia, invasive squamous cell carcinoma, large cell carcinoma, leiomyosarcoma, lentigo maligna melanoma, malignant melanoma, malignant mesothelioma, medulloblastoma, medulloepithelioma, and combinations thereof, but is not limited thereto.

In addition, the present invention provides a pharmaceutical composition for combination administration for preventing or treating cancer or a cell hyperplasia disorder, including the nanoparticles and an immune anticancer agent as effective ingredients.

In the present invention, “immune anticancer agent” means an agent that kills cancer cells by activating the human immune system, and includes immune checkpoint inhibitors, immune cell therapies, therapeutic antibodies, anticancer virus therapies, cancer treatment vaccines, immunomodulators, and the like, but is not limited thereto. The immune anticancer agent is preferably an immune checkpoint inhibitor, and more preferably may be any one or more selected from the group consisting of anti-PD-L1 (programmed death-ligand 1) antibody, anti-CTLA-4 (cytotoxic T-lymphocyte antigen-4) antibody, anti-PD-1 (programmed death-1) antibody, and combinations thereof, but is not limited thereto.

Additionally, the present invention provides a method of preventing or treating cancer or a cell hyperplasia disorder, including administering the nanoparticles to a subject.

In the present invention, the term “subject” is not limited to a mammal such as livestock or a human that requires prevention, treatment, and/or diagnosis of the disease, but may be preferably a human.

The term “administration” of the present invention means providing a predetermined substance to a patient by any appropriate method, and the pharmaceutical composition of the present invention can be formulated in various forms for administration to a subject, and a representative formulation for parenteral administration is an injectable formulation, preferably an isotonic aqueous solution or suspension. Injectable formulations can be prepared according to techniques known in the art using suitable dispersing or wetting agents and suspending agents. For example, each component can be dissolved in saline or a buffer solution and formulated for injection. In addition, formulations for oral administration include, for example, ingestible tablets, buccal tablets, troches, capsules, elixirs, suspensions, syrups, and wafers, and these formulations may contain, in addition to effective ingredients, diluents (e.g., lactose, dextrose, sucrose, mannitol, sorbitol, cellulose and/or glycine) and lubricants (e.g., silica, talc, stearic acid and its magnesium or calcium salts and/or polyethylene glycol). The tablets may contain binding agents such as magnesium aluminum silicate, starch paste, gelatin, tragacanth, methylcellulose, sodium carboxymethylcellulose and/or polyvinylpyrrolidine, and may optionally further contain disintegrants such as starch, agar, alginic acid or its sodium salt, absorbents, colorants, flavoring agents and/or sweetening agents. The formulations may be prepared by conventional mixing, granulation or coating methods.

The pharmaceutical composition according to the present invention may be administered through various routes including oral, transdermal, subcutaneous, intravenous or intramuscular routes, and the dosage of the active ingredient may be appropriately selected according to various factors such as the route of administration, the patient's age, sex, weight, and the severity of the patient's condition. In addition, the composition of the present invention may be administered in combination with known compounds that can enhance the desired effect.

As routes of administration for the pharmaceutical composition according to the present invention, the pharmaceutical composition may be administered to humans and animals orally or parenterally such as intravenously, subcutaneously, intranasally, or intraperitoneally. Oral administration also includes sublingual application. Parenteral administration includes injection methods such as subcutaneous injection, intramuscular injection, and intravenous injection, and instillation methods.

The terminology used herein is for the purpose of describing particular embodiments only and is not to be limiting of the embodiments. The singular form is intended to include the plural forms as well, unless the context clearly indicates otherwise. It will be further understood that the terms “comprise” or “have” when used herein, specify the presence of stated features, integers, steps, operations, elements, components, or combinations thereof, but do not preclude the presence or addition of one or more other features, integers, steps, operations, elements, components, or combinations thereof.

Unless otherwise defined, all terms including technical and scientific terms used herein have the same meaning as commonly understood by one of ordinary skill in the art to which the embodiments belong. It will be further understood that terms, such as those defined in commonly-used dictionaries, should be interpreted as having a meaning that is consistent with their meaning in the context of the relevant art and will not be interpreted in an idealized or overly formal sense unless expressly so defined herein.

Terms such as first, second, A, B, (a), (b), or the like may be used to describe components of the embodiments. These terms are used only for the purpose of discriminating one component from another component, and the nature, the sequences, or the orders of the components are not limited by the terms. It should be noted that if one component is described as being “connected,” “coupled” or “joined” to another component, the former may be directly “connected,” “coupled,” and “joined” to the latter or “connected”, “coupled”, and “joined” to the latter via another component.

### MODE FOR CARRYING OUT THE INVENTION

Hereinafter, embodiments will be described in detail with reference to the accompanying drawings. However, various alterations and modifications may be made to the embodiments. Here, the embodiments are not construed as limited to the disclosure. The embodiments should be understood to include all changes, equivalents, and replacements within the technical scope of the disclosure.

When describing the embodiments with reference to the accompanying drawings, like reference numerals refer to like components and a repeated description related thereto will be omitted. In the description of embodiments, detailed description of well-known related technology will be omitted when it is deemed that such description will cause ambiguous interpretation of the present invention.

The present invention can be modified in various ways and has various embodiments. Specific embodiments are illustrated in the drawings and described in detail in the detailed description below. However, this is not intended to limit the present invention to specific embodiments, but should be understood to include all changes, equivalents, or substitutes included in the idea and technical scope of the present invention. In the description of the present invention, detailed description of well-known related technology will be omitted when it is deemed that such description will cause ambiguous interpretation of the present invention.

### Example 1. Preparation of Nanoparticles of the Present Invention

Methylene blue and a cationic adjuvant were respectively modified in the form of a methylene blue-organic acid complex and a cationic adjuvant-organic acid complex. Subsequently, nanoparticles were prepared through an oil-in-water technique using a polymer capable of clinical intravenous injection with the methylene blue-organic acid complex and the cationic adjuvant-organic acid complex.

### Example 1.1. Preparation of Methylene Blue-Oleic Acid Complex

20 mg of methylene blue (MB, purchased from Aldrich) and 30 mg of sodium oleate (O, purchased from Aldrich) were heated in 100 mL of tetrahydrofuran (THE, Daejung Chemicals) at 60 to 90° C. for 1 to 5 minutes to dissolve methylene blue. Methylene blue formed a stable methylene blue-oleic acid complex (MBO) with sodium oleate by electrostatic force and hydrophobic interactions in molecular structure, thereby exhibiting high solubility in the solvent. The mixture in which methylene blue was dissolved in this manner was filtered using a syringe filter (5 μm) to remove excess sodium oleate and other impurities, and then the solvent was evaporated to obtain MBO.

### Example 1.2. Preparation of Imiquimod-Oleic Acid Complex

First, to increase drug encapsulation by surfactants, imiquimod (Im) was chemically complexed with sodium oleate (O, purchased from Aldrich) through ionic complexation. Specifically, 10 mg of imiquimod was weighed and added to distilled water (DW) at a concentration of 1 mg/ml. Subsequently, HCl (37% solution) was added to imiquimod in water at a ratio of 1:1, and the mixture was ultrasonicated until imiquimod, which is generally insoluble in water, was completely dissolved. Separately, 13 mg of sodium oleate was weighed and dissolved in water, then added to the protonated imiquimod at a 1:1 ratio to form an imiquimod-oleic acid complex (ImO) (Method 1), which showed a turbid solution. The final concentration of the ImO complex was 0.5 mg/ml.

### Example 1.3. Preparation of Nanoparticles of the Present Invention

To prepare the nanoparticles of the present invention (ImoMB), a methylene blue-oleic acid complex (MBO) and Pluronic F-127 (Pluronic® F127) thin film were obtained.

First, 20 mg of Pluronic F-127 was weighed in a glass vial and dissolved in tetrahydrofuran (THF, Daejung Chemicals). Next, 50 μl of 1 mg/ml MBO stock solution obtained in Example 1.1 was added to Pluronic F-127 in THE, followed by vortexing and ultrasonication. Subsequently, the solvent was evaporated by air blowing to form a thin film on the wall of the glass vial. The thin film was rehydrated with 1 ml of a 0.5 mg/ml ImO complex solution and ultrasonicated. After ultrasonication, the solution was transferred to a flask tube and shaken for at least 2 hours while being protected from light so that the ImO complex was completely encapsulated into nanoparticles to synthesize ImoMB.

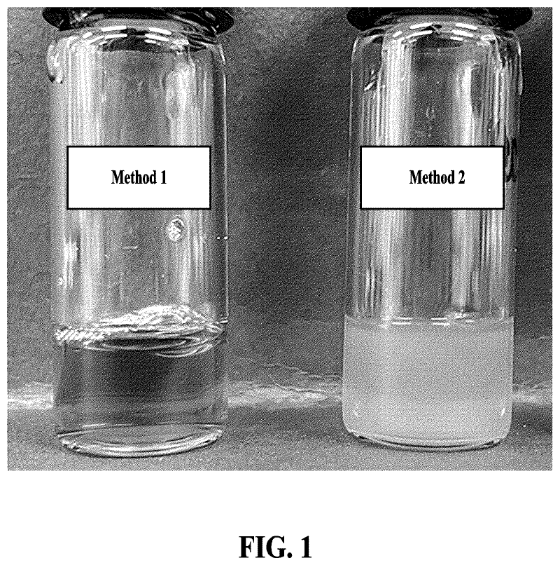

On the other hand, when oleic acid was added to water in which imiquimod was dispersed without using HCl to obtain protonated imiquimod in Example 1.2 (Method 2), it was confirmed that dispersibility was significantly reduced when forming particles with ImoMB (FIG. 1). Therefore, the nanoparticles of the present invention were prepared according to Method 1.

### Comparative Example 1. Preparation of Nanoparticles Not Loaded with Imiquimod (NanoMB)

Nanoparticles (NanoMB) were synthesized in the same manner as Example 1.3 using a methylene blue-oleic acid complex (MBO) and Pluronic F-127 without loading imiquimod, and 1 ml of distilled water (DW) was used instead of ImO complex solution in the rehydration step of the thin film.

### Comparative Example 2. Preparation of Nanoparticles in which Imiquimod was Not Modified with Organic Acid (ImiMB)

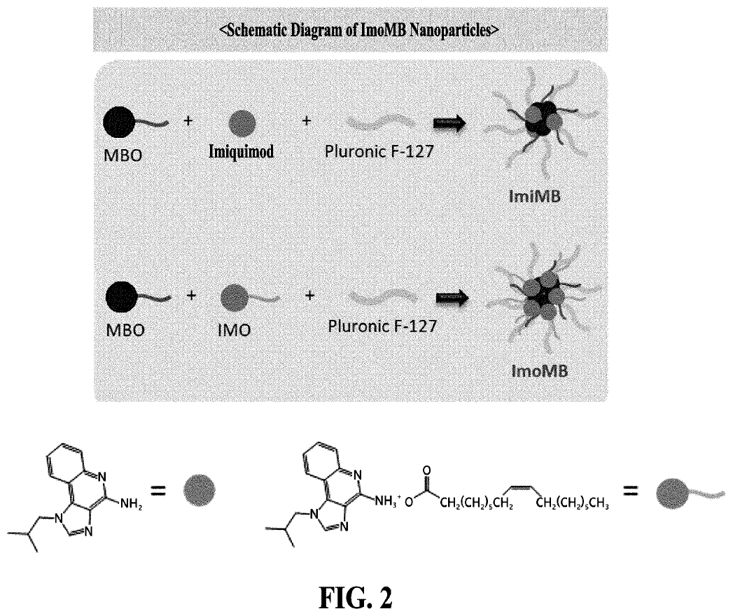

Nanoparticles (ImiMB) were synthesized using a methylene blue-oleic acid complex (MBO) and Pluronic F-127 with imiquimod in a free imiquimod state without modifying imiquimod with oleic acid. Since free imiquimod is not soluble in water, the free imiquimod was added together during the formation of the MBO and Pluronic F127 thin film in Example 1.3. Subsequently, 1 ml of distilled water (DW) was used instead of ImO complex solution in the rehydration step of the thin film (FIG. 2).

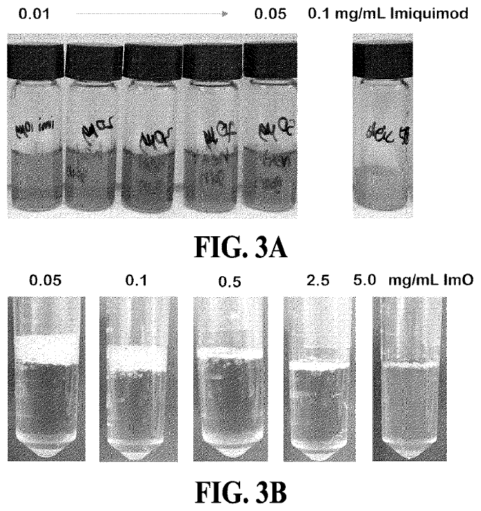

On the other hand, in the case of free imiquimod, the free imiquimod did not dissolve even at a low concentration of 0.1 mg/ml, but in the case of imiquimod-oleic acid complex (ImO), it was confirmed that particle formation occurred well even at a high concentration of 5.0 mg/ml, maintaining a transparent solution form (FIG. 3). Therefore, in the present invention, imiquimod was modified with an organic acid to form a cationic adjuvant-organic acid complex for solubility improvement and high-content loading, and then nanoparticles were prepared using the cationic adjuvant-organic acid complex.

### Comparative Example 3. Preparation of Mixture of NanoMB and ImO Nanoparticles (ImO-F127)

Nanoparticles (NanoMB) prepared by adding Pluronic F-127 to the methylene blue-oleic acid complex (MBO) prepared according to Example 1.1 and nanoparticles (ImO-F127) prepared by adding Pluronic F-127 to the imiquimod-oleic acid complex (ImO) prepared according to Example 1.2 were simply mixed.

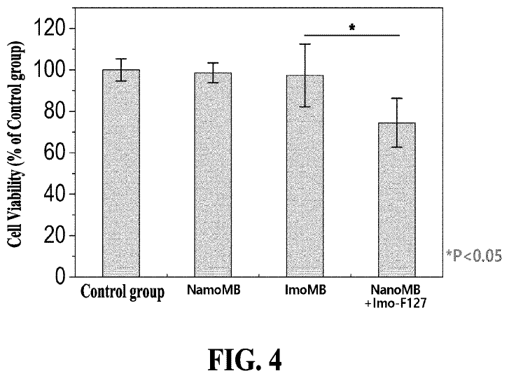

In the case of the mixture, unlike the nanoparticles of the present invention (ImoMB) and nanoparticles not loaded with imiquimod (NanoMB), the mixture showed toxicity to normal cells (FIG. 4).

### Experimental Example 1. Characteristics of Nanoparticles of the Present Invention

### Experimental Example 1.1. Shape and Size of Nanoparticles of the Present Invention

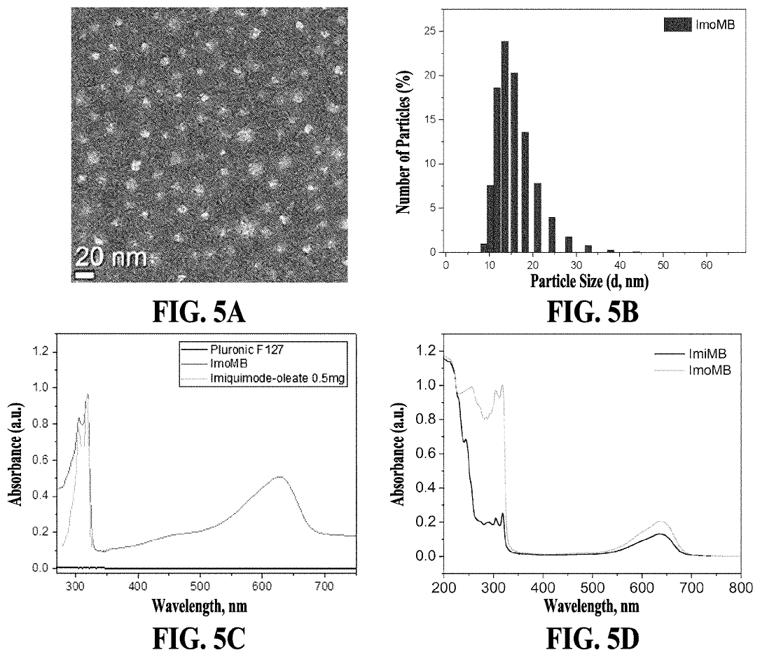

The shape of the nanoparticles of the present invention (ImoMB) prepared in Example 1 was observed using transmission electron microscopy (TEM, CM30, PEI/Philips, 200 kV), and the size of the nanoparticles was measured using Zetasizer Nano ZSo (Malvern Instruments, UK). As a result, it was confirmed that the nanoparticles were spherical particles having an average diameter of 15 nm (FIGS. 5A and 5B).

### Experimental Example 1.2. Absorbance of Nanoparticles of the Present Invention

The absorbance of imiquimod-oleic acid complex (ImO) dissolved in water and the nanoparticles of the present invention (ImoMB) was measured. As a result, it was confirmed that the absorption wavelength of imiquimod appeared in the short wavelength region (FIG. 5C).

In addition, as a result of comparing the absorbance of nanoparticles in which imiquimod was not modified with oleic acid (ImiMB) and the nanoparticles of the present invention, the nanoparticles of the present invention showed 5 times higher imiquimod absorbance (FIG. 5D), which means that the nanoparticles of the present invention can load 5 times more imiquimod.

### Experimental Example 2. Phototoxicity of Nanoparticles of the Present Invention

To confirm the cytotoxicity of the nanoparticles of the present invention (ImoMB), 100 μl of cell culture medium in which 1×10⁴ cells were dispersed was placed in a cell culture dish (96-well plate) and cultured in an incubator (5% CO₂, 37° C.) for 24 hours, washed with DPBS, and then a mixture of 10 μl of the nanoparticles at 20.55 mg/ml and 90 μl of cell culture medium was added to the cells and cultured in the same incubator for 1 hour. After washing with DPBS, the number of each cell was observed through MTT assay. In addition, to evaluate the phototoxicity of the nanoparticles against each cancer cell, the experiment was conducted in the same manner as the above experiment, except that MTT analysis was performed after 10 minutes of laser irradiation for each cell to confirm the number of cells due to phototoxicity. The experiment was performed on head and neck cancer cell lines SCC7 and FADU.

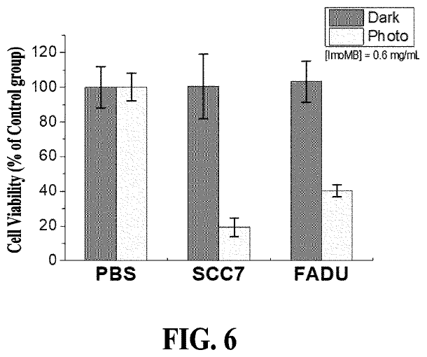

As a result, no toxicity was shown in the case of no light irradiation (dark conditions), but significant toxicity was confirmed in the case of 633 nm light irradiation (light conditions) (FIG. 6). That is, it can be seen that the nanoparticles of the present invention increase danger and damage signal expression against cancer cells under light conditions.

### Experimental Example 3. Cell Death of Nanoparticles of the Present Invention Upon Light Irradiation

To confirm whether the nanoparticles of the present invention (ImoMB) cause cell death under light conditions, damage-associated molecular patterns (DAMP) that cause inflammatory reactions in cells and induce immune responses were confirmed.

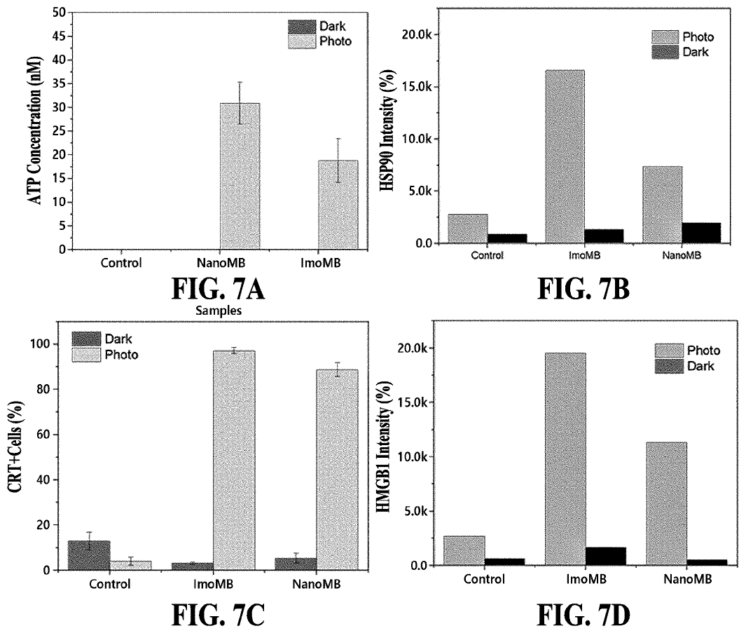

As a result, compared to the control group, not only ATP concentration but also the expression of HSP90, calreticulin (CRT), and HMGB1 all greatly increased, and in particular, HSP90, CRT, and HMGB1 showed higher expression levels than nanoparticles not loaded with imiquimod (NanoMB) (FIG. 7).

This suggests that the nanoparticles of the present invention are not toxic under biological conditions but are photodynamic therapeutic agents that can induce selective death of target cells upon light irradiation.

### Experimental Example 4. Evaluation of Dendritic Cell Activation Function of Nanoparticles of the Present Invention

To confirm the immune activation effect by the nanoparticles of the present invention (ImoMB), the degree of maturation of immature dendritic cells obtained from bone marrow cells isolated from mice and the lymph and spleen of male mice (C3H, 5 weeks old, Orient Bio) was confirmed. At this time, an untreated control group, an LPS-treated group as a positive control group, a group treated with nanoparticles not loaded with imiquimod (NanoMB), a group treated with a simple mixture of NanoMB and free imiquimod, and a free imiquimod alone-treated group as comparative examples were prepared.

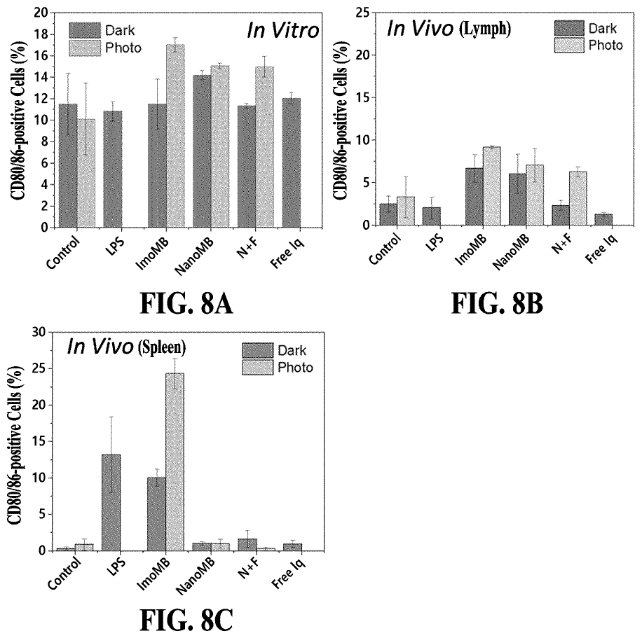

As a result, it was confirmed that the nanoparticles of the present invention induced significantly increased levels of mature dendritic cells compared to the control group at both cellular and animal levels. In particular, it was confirmed that more dendritic cells were activated at both cellular and animal levels compared to the NanoMB-treated group, the group treated with the simple mixture of NanoMB and free imiquimod, and the free imiquimod alone-treated group (FIG. 8).

This suggests that the nanoparticles of the present invention are adjuvants that can very effectively induce immune activation in vivo.

### Experimental Example 5. Anticancer Effect of Nanoparticles of the Present Invention in Animal Model

60 μl of culture medium in which 1×10⁷ SCC7 cells of head and neck cancer cell line were dispersed was injected into the left gluteal muscle area of male mice (C3H, 5 weeks old, Orient Bio), and after visually confirming the growth of cancer tissue for 2 weeks, 60 μl of the nanoparticles of the present invention (ImoMB) was subcutaneously injected into the cancer tissue area to observe photodynamic and immunotherapy effects against primary cancer. At this time, an untreated control group (PBS), a group treated with nanoparticles not loaded with imiquimod (NanoMB), a group treated with a simple mixture of NanoMB and free imiquimod, and a free imiquimod alone-treated group as comparative examples were prepared.

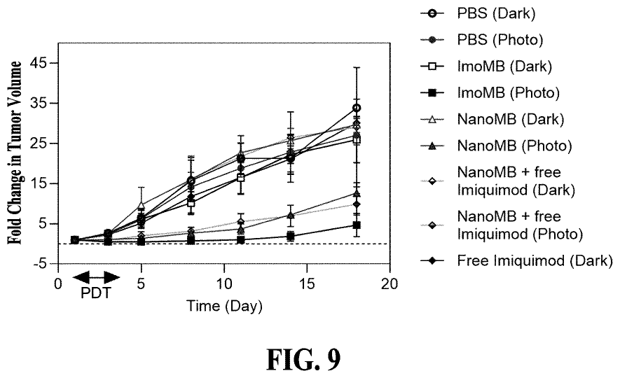

As a result, it was confirmed that the nanoparticles of the present invention significantly reduced tumor volume upon light irradiation. In particular, the nanoparticles showed significantly improved therapeutic effects compared to the NanoMB-treated group, the group treated with the simple mixture of NanoMB and free imiquimod, and the free imiquimod alone-treated group (FIG. 9).

This suggests that the nanoparticles of the present invention show enhanced anticancer effects against primary cancer through photodynamic and immunotherapy.

### Experimental Example 6. Secondary Tumor Growth Inhibition Effect of Nanoparticles of the Present Invention

60 μl of culture medium in which 1×10⁷ SCC-7 cells of head and neck cancer cell line were dispersed was injected into the left and right gluteal muscle areas of male mice (C3H, 5 weeks old, Orient Bio), respectively, and after visually confirming the growth of cancer tissue for 2 weeks, 60 μl of the nanoparticles of the present invention (ImoMB) was subcutaneously injected into the left cancer tissue area. Thereafter, changes in right tumor volume were observed after 14 days. At this time, an untreated control group and a group treated with nanoparticles in which imiquimod was not modified with oleic acid (ImiMB) as a comparative example were prepared.

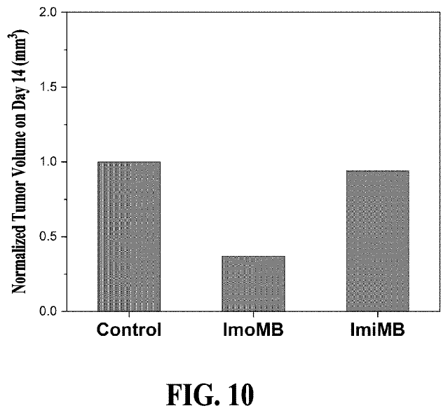

As a result, the ImiMB-treated group showed no significant difference from the control group, but it was confirmed that the nanoparticle-treated group of the present invention showed a significant decrease in secondary tumor volume by more than half (FIG. 10). That is, it can be seen that the nanoparticles of the present invention inhibit the growth of secondary tumors through memory effects in vivo.

### Experimental Example 7. Anticancer Efficacy Upon Combination Administration with Immune Checkpoint Inhibitor

60 μl of culture medium in which 1×10⁷ SCC-7 cells of head and neck cancer cell line were dispersed was injected into the left gluteal muscle area of male mice (C3H, 5 weeks old, Orient Bio), and after visually confirming the growth of cancer tissue for 2 weeks, 60 μl of the nanoparticles of the present invention (ImoMB) was subcutaneously injected into the cancer tissue area. Subsequently, after photodynamic therapy (655 nm laser irradiation for 10 minutes) for a total of 3 times at 2-day intervals, anti-PD-L1 antibody, which is an immune checkpoint inhibitor, was added a total of 3 times at 3-day intervals. Thereafter, changes in tumor size over time were observed for about 10 days.

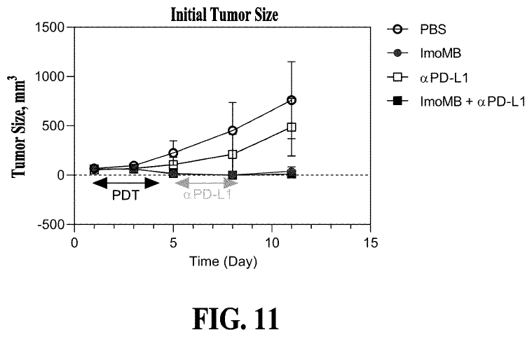

As a result, compared to the untreated control group or when anti-PD-L1 was treated alone, when the nanoparticles of the present invention were treated or when the nanoparticles of the present invention and anti-PD-L1 antibody were administered in combination, it was confirmed that tumor growth was significantly inhibited (FIG. 11).

In addition, cytokine expression in blood was confirmed in addition to changes in tumor size. After observing tumor size for about 10 days, blood was separated from experimental mice, and cytokines related to anticancer immunity in blood were quantified by enzyme immunoassay.

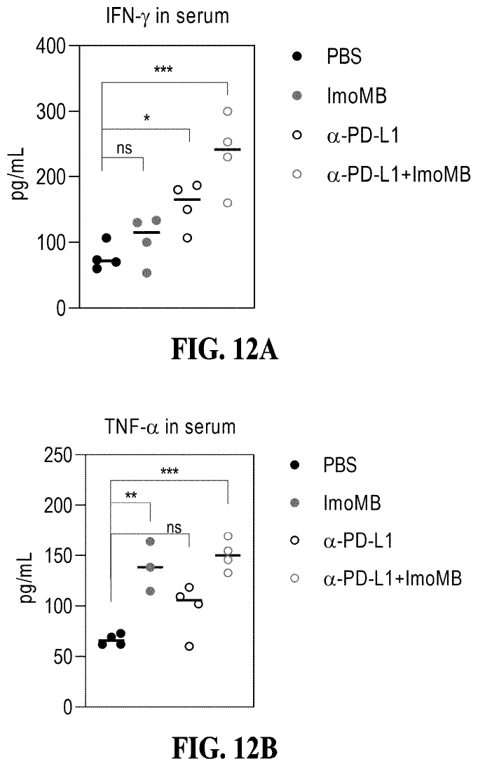

As a result, compared to when anti-PD-L1 was treated alone or when the nanoparticles of the present invention were treated alone, a significant increase in cytokine expression such as IFN-γ and TNF-α was observed (FIG. 12). This suggests that due to the synergistic effect between the nanoparticles of the present invention and anti-PD-L1, cellular immunity or anticancer immunity is further increased.

While the embodiments have been described with reference to the limited drawings, it will be apparent to one of ordinary skill in the art that various alterations and modifications can be made from the above description. For example, suitable results may be achieved if the described techniques are performed in a different order, and/or if components in a described system, architecture, device, or circuit are combined in a different manner, or replaced or supplemented by other components or their equivalents.

Therefore, other implementations, other embodiments, and equivalents to the claims are also within the scope of the following claims.

The present invention was completed with support from the Korea Institute of Science and Technology's institutional unique project 2V08711: Development of photoimmunotherapy technology for head and neck cancer, and national research and development projects 2N72040: Development of next-generation anticancer technology based on bacteria shuttle-induced nanomaterials and peptide light sources (Project No. 2022M3H4A1A03076638) and 2N69690: Development of nanocomposite anticancer vaccine based on photosensitive supramolecular immune switching (Project No. 2021R1A2C2005418).

1. A nanoparticle comprising:
a methylene blue-organic acid complex;
a cationic adjuvant-organic acid complex; and
an amphiphilic polymer.

2. The nanoparticle of claim 1, wherein the cationic adjuvant is an imidazoquinoline compound comprising an amine functional group.

3. The nanoparticle of claim 2, wherein the imidazoquinoline compound comprising the amine functional group is any one or more selected from imiquimod, resiquimod, gardiquimod, and combinations thereof.

4. The nanoparticle of claim 1, wherein the organic acid is any one or more acids or salts thereof selected from the group consisting of propionic acid, butyric acid, pentanoic acid, hexanoic acid, heptanoic acid, octanoic acid, oleic acid, linoleic acid, linolenic acid, stearic acid, palmitic acid, salicylic acid, cholic acid, deoxycholic acid, lithocholic acid, and combinations thereof.

5. The nanoparticle of claim 1, wherein the amphiphilic polymer is any one or more selected from the group consisting of polyethylene oxide-polypropylene oxide-polyethylene oxide (PEO-PPO-PEO) block copolymer, polylactic acid (PLA), polyglycolic acid (PGA), block copolymer of poly(lactic-co-glycolic acid) (PLGA) and polyethylene glycol (PEG), and combinations thereof.

6. The nanoparticle of claim 1, wherein the methylene blue-organic acid complex and the cationic adjuvant-organic acid complex are encapsulated inside amphiphilic polymer micelles.

7. The nanoparticle of claim 1, wherein the methylene blue-organic acid complex comprises methylene blue complexed with an organic acid.

8. The nanoparticle of claim 1, wherein the cationic adjuvant-organic acid complex is imiquimod physically modified with an organic acid.

9. A pharmaceutical composition for preventing or treating cancer or a cell hyperplasia disorder, comprising the nanoparticle of claim 1 as an effective ingredient.

10. The pharmaceutical composition of claim 9, wherein the nanoparticle is used simultaneously for photodynamic therapy and immunotherapy.

11. The pharmaceutical composition of claim 9, wherein the nanoparticle activates dendritic cells to selectively kill cancer cells.

12. The pharmaceutical composition of claim 9, wherein the cancer is any one or more selected from the group consisting of skin cancer, breast cancer, uterine cancer, esophageal cancer, gastric cancer, brain tumor, colon cancer, rectal cancer, colorectal cancer, lung cancer, ovarian cancer, cervical cancer, endometrial cancer, vulvar cancer, kidney cancer, blood cancer, pancreatic cancer, pancreatic endocrine tumor, prostate cancer, testicular cancer, nasopharyngeal cancer, laryngeal cancer, head and neck cancer, thyroid cancer, liver cancer, bladder cancer, thymoma, mesothelioma, osteosarcoma, lymphoma, leukemia, thymic cancer, urethral cancer, bronchial cancer, gallbladder cancer, penile cancer, ureteral cancer, renal cell carcinoma, non-Hodgkin's lymphoma, myelodysplastic syndrome, multiple myeloma, plasma cell tumor, pediatric cancer, and combinations thereof.

13. The pharmaceutical composition of claim 9, wherein the cell hyperplasia disorder is any one or more selected from the group consisting of acral lentiginous melanoma, actinic keratosis, adenocarcinoma, adenoid cystic carcinoma, adenoma, adenosarcoma, adenosquamous carcinoma, astrocytic tumor, glucagonoma, hemangioblastoma, hemangioendothelioma, hemangioma, hepatic adenoma, hepatic adenomatosis, hepatocellular carcinoma, insulinoma, intraepithelial neoplasia, intraepithelial squamous cell neoplasia, invasive squamous cell carcinoma, large cell carcinoma, leiomyosarcoma, lentigo maligna melanoma, malignant melanoma, malignant mesothelioma, medulloblastoma, medulloepithelioma, and combinations thereof.

14. The pharmaceutical composition of claim 9, wherein the pharmaceutical composition further comprises an immune anticancer agent.

15. The pharmaceutical composition of claim 14, wherein the immune anticancer agent is any one or more selected from the group consisting of anti-PD-L1 antibody, anti-CTLA-4 antibody, anti-PD-1 antibody, and combinations thereof.

16. A topical dosage form for preventing or treating cancer or a cell hyperplasia disorder, comprising the nanoparticle of claim 1.

17. A method of preparing nanoparticles, comprising the following steps:
(1) adding an organic acid to methylene blue to form a methylene blue-organic acid complex;
(2) adding an organic acid to a cationic adjuvant to form a cationic adjuvant-organic acid complex;
(3) adding the methylene blue-organic acid complex formed in step (1) to an amphiphilic polymer and performing ultrasonic treatment to form a thin film;
(4) rehydrating the thin film formed in step (3) with the cationic adjuvant-organic acid complex formed in step (2) and performing ultrasonic treatment; and
(5) encapsulating a mixture of step (4) to prepare nanoparticles.
